(19) United States
(12) Patent Application Publication
(10) Pub. No.: US 2017/0184872 A1
(43) Pub. Date: Jun. 29, 2017

(54) Interchangeable Eyeglass Lens Rim Using Magnetic Retaining Forces

(71) Applicant: Max Aaron Silver, Boca Raton, FL (US)

(72) Inventor: Max Aaron Silver, Boca Raton, FL (US)

(21) Appl. No.: 14/572,997

(22) Filed: Dec. 17, 2014

(51) Int. Cl. G02C 1/00; G02C 9/00; G02C 7/02; A61F 9/02; G02C 1/02

(52) U.S. Cl. CPC G02C 1/10; A61F 9/025; G02C 1/02; G02C 7/02; G02C 9/00; G02C 2200/02; G02C 2200/08

(57) ABSTRACT

An eyeglass lens rim and frame system having an interchangeable lens rim is disclosed. The eyeglass lens element comprises a pair of eyeglass lenses, each fully encircled by a lens rim, and incorporating at least one magnetic element within the lens rim. The eyeglass frame element further comprises at least one receiving section or channel into which the eyeglass lens rim element precisely fits, with the eyeglass frame element also incorporating at least one magnetic element within the frame to attract and maintain the eyeglass lens rim element, and magnetic element incorporated therein, in specific position within the at least one receiving section of the eyeglass frame element. The interchangeability of the entire lens rim provides a ready means of quickly and fully altering the appearance of the eyeglasses instead of merely replacing the lens pieces.

### FIELD OF THE INVENTION

The present invention generally relates to eyeglass frames and lens rims, and more particularly relates to eyeglass lens rims that are readily interchangeable with an eyeglass frame section such that the lens rim is held in proper position with respect to the eyeglass frame section using, at least, a channel section and magnetic retaining elements incorporated into one or both of the lens rim and eyeglass frame. The eyeglass frame and lens rim may be manufactured from a variety of materials including various thermoplastics or metals, the latter including aluminum or titanium. With the ability to easily interchange or substitute eyeglass lenses and lens rims used with the eyeglass frames, the entire appearance of the eyeglasses, and thus the appearance of the wearer, can be readily altered, allowing the wearer to quickly change his or her “look and feel” for different activities, events, or different times of the day or night.

### BACKGROUND OF THE INVENTION

Eyeglasses or glasses are worn by millions of people around the world, whether for normal sight correction, for close-up reading, for protection from the sun or snow glare, or simply for the way they make the wearer look. Many, if not most people, have multiple sets of eyeglasses that can be worn for a variety of uses or activities, including for everyday use, for reading, and for outdoor use and activities. Often, eyeglass wearers have different designs and styles of frames and lenses that allow the wearer to have a different “look” based upon which eyeglasses are worn for particular activities. While eyeglass wearers may have multiple sets of glasses to wear for different purposes or activities, they often have one particular set of glasses that are most comfortable, either because of the design, the frame shape, the shape and orientation of the temple/side pieces, or the design and comfort of the nose cushions or nose bridge.

To accommodate the desire of wearers to have different eyeglass designs, various interchangeable or replacement eyeglass lenses have been created over the years. Several of the designs have been manufactured, and some of which are currently being commercialized.

Two examples includes U.S. Pat. Nos. 8,641,188 and 7,850,301, both for Eyewear Frames With Magnetic Lens Attachments and both issued to DiChiara (the DiChiara patents). The '188 DiChiara patent and the '301 DiChiara patent each disclose a pair of eyeglass lenses that are attachable to an eyeglass frame using magnets or magnetically attractive material. The description and disclosure provided in the DiChiara patents provides for the magnets to be incorporated into each individual lens such that each lens is attached and removed from the eyeglass frame individually. Such a design does not provide any means for changing the style, shape or look of the eyeglasses through use of a lens rim that maintains the lenses.

Another example of a system is shown in U.S. Pat. No. 7,661,815 for a Eyeframe With Interchangeable Lenspieces Held by a Magnetic Closure and Interchangeable Lens System, issued to Lipawsky (the “'815 patent”). The '815 patent describes a mechanism for interchanging lenspieces within a rimwire eyeglass frame that uses a magnetic closure and rotating center section to hold the lenspieces in place. The rotating center section of the rimwire eyeglass frame is susceptible to breakage and the process for manufacturing such a device is fairly complex.

A further system, that is being commercialized is shown in U.S. Pat. No. 5,642,177 for Detachable Sunglasses with Magnets issued to Nishioka (the '377 patent”). As described in the '377 patent, the sunglasses are attached to the eyeglass frame, both having the same shape by a center located magnet. The design of the '377 patent is to provide sunglasses to be used by the wearer who has prescription glasses and does not wish to have a separate set of prescription sunglasses. There is no suggestion in the '377 patent for a system or lens mechanism that allows the wearer to alter the shape, style or look of his or her glasses over simply making them sunglasses.

As described, none of these above designs and products fully addresses the desire of wearers to readily substitute the lens rim of a set of eyeglasses, while still using the same eyeglass frame, and provide a means to alter the entire look, aesthetics, and style of the eyeglasses. Moreover, many of the prior art devices and interchangeable eyeglasses are difficult to manufacture and/or are difficult and clumsy use or operate. To be useful, such devices must not only be easily manufactured, but must also be available in a variety of materials, shapes, styles, and colors, in order to achieve market success and meet the objective of allowing the user to readily alter his or her “look.” Accordingly, such prior devices have not achieved market wide success or utility.

What is needed is an interchangeable eyeglass system that allows for ready substitution of the lens rim, including the two lenses, into a fixed eyeglass frame, such that with the interchangeability of the lens rim and associated lenses, the entire look and image of the eyeglasses may be altered. Such a design using both magnetic retaining elements and a channel section within the eyeglass frame and/or lens rim has not been created or achieved in the prior art. Such an eyeglass system would allow wearers to easily change their entire “look” with an easy interchanging of eyeglass rim and lenses.

### SUMMARY OF THE INVENTION

The above described problems that are inadequately or incompletely resolved by prior art designs, are addressed and solved by the present invention.

A preferred embodiment of the invention is an interchangeable eyeglass lens rim and eyeglass frame system, said system comprising (a) a lens rim encircling and holding a pair of eyeglass lenses; (b) at least one magnetic element incorporated within said lens rim; (c) an eyeglass frame component having a channel section along one edge into which said lens rim fits; and (d) at least one magnetic element located within said eyeglass frame component such that said at least one magnetic element attracts said at least one magnetic element within said lens rim, and thereby firmly maintains said lens rim in position within said channel section and against said eyeglass frame component.

Another embodiment of the invention is an eyeglass frame and lens rim set wherein said lens rim is interchangeable, said frame and lens rim set comprising (a) an eyeglass frame component having a cut out section along a lower edge; (b) at least a first magnetic section incorporated within said cut out section of said eyeglass frame; (c) a lens rim component fully encircling a pair of eyeglass lenses; (d) at least a second magnetic section incorporated along a top edge of said lens rim component; whereby with said lens rim component properly positioned within said eyeglass frame component and within said cut out section, said second magnetic section attracts said first magnetic section and holds said lens rim component firmly in place with said eyeglass frame component in said cut out section.

A further embodiment of the invention is an interchangeable eyeglass lens rim and eyeglass frame system, said system comprising (a) a lens rim encircling and holding a pair of eyeglass lenses, said lens rim having a channel section along said lens rim upper side; (b) at least one magnetic element incorporated within said lens rim; (c) an eyeglass frame component having a lower side that securely fits into said channel section of said lens rim; and (d) at least one magnetic element located within said eyeglass frame component such that said at least one magnetic element attracts said at least one magnetic element within said lens rim, and thereby firmly maintains said eyeglass frame component in position within said channel section and against said lens rim.

The invention will be best understood by a review of the following detailed description of preferred embodiments in conjunction with the drawings briefly described below.

### BRIEF DESCRIPTION OF THE DRAWINGS

For the purposes of illustrating the invention, the attached drawings show several embodiments that are presently preferred. However, it should be understood that the invention is not limited to the precise arrangement and instrumentality shown in the accompanying drawings which are several embodiments.

FIG. 1: is a front view of an exemplary embodiment of the interchangeable eyeglass lens rim connected to the eyeglass frame;

FIG. 1A: is a front view of an exemplary embodiment of the interchangeable eyeglass lens rim separated from the eyeglass frame;

FIG. 2: is an exploded front view of an exemplary embodiment of the interchangeable eyeglass lens rim separated from the eyeglass frame;

FIG. 3: is a front view of another exemplary embodiment of the interchangeable eyeglass lens rim and eyeglass frame showing placement of additional magnetic sections;

FIG. 3A: is a front view of an exemplary embodiment of the interchangeable eyeglass lens rim with additional magnetic sections, and separated from the eyeglass frame;

FIG. 4: is a top view of an exemplary embodiment of the interchangeable eyeglass lens rim and eyeglass frame with the lens rim section in proper position;

FIG. 4A: is a top view of an exemplary embodiment of the interchangeable eyeglass lens rim and eyeglass frame with the lens rim section in proper position showing an exemplary placement of frame ridge sections and associated lens rim notches;

FIG. 5: is a cut-away exploded view of one side of an exemplary embodiment of the eyeglass frame and channel into which the lens rim and lenses fit;

FIG. 6: is a front view of a first exemplary embodiment of three shapes of interchangeable lens rims in position in an associated eyeglass frame;

FIG. 7: is a front view of a second exemplary embodiment of three shapes of interchangeable lens rims in position in an associated eyeglass frame; and

FIG. 8: is a front view of a third exemplary embodiment of three shapes of interchangeable lens rims in position in an associated eyeglass frame.

### DETAILED DESCRIPTION OF PREFERRED EMBODIMENTS

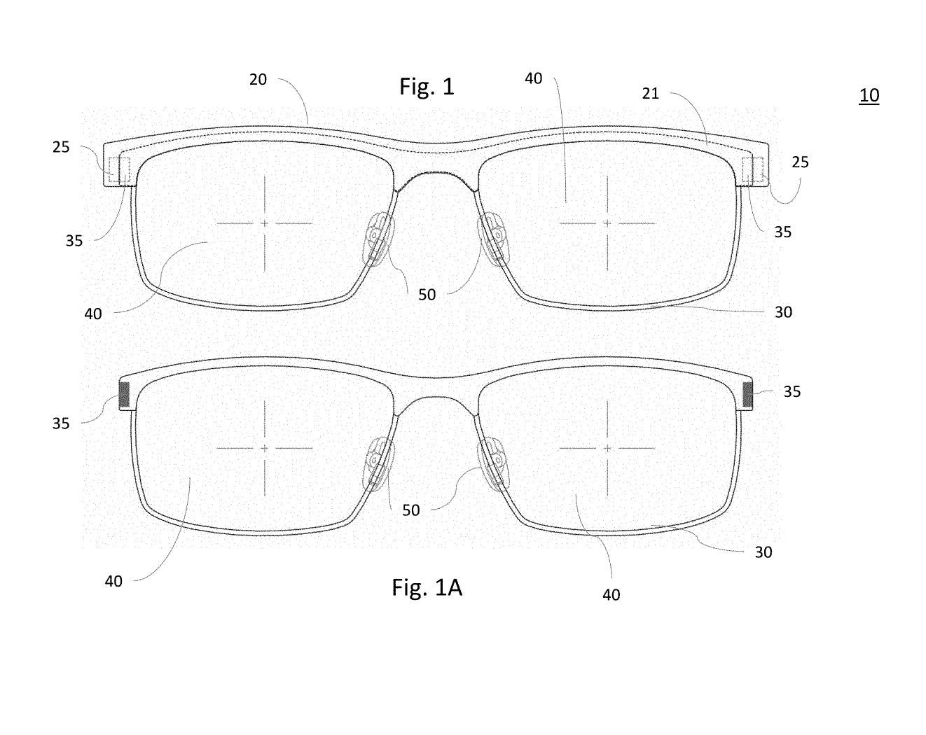

An interchangeable eyeglass lens rim and eyeglass frame system is described and disclosed. The primary elements of the eyeglass lens rim and frame system 10, as illustrated in one embodiment in FIGS. 1 and 1A, include an eyeglass frame section 20, an eyeglass lens rim 30, two eyeglass lenses 40, at least one magnetic retaining section 25 incorporated into the eyeglass frame section 20, a channel or groove section 21 incorporated into the lower side of the eyeglass frame section 20, and an associated at least one magnetic retaining section 35 incorporated into the eyeglass lens rim 30.

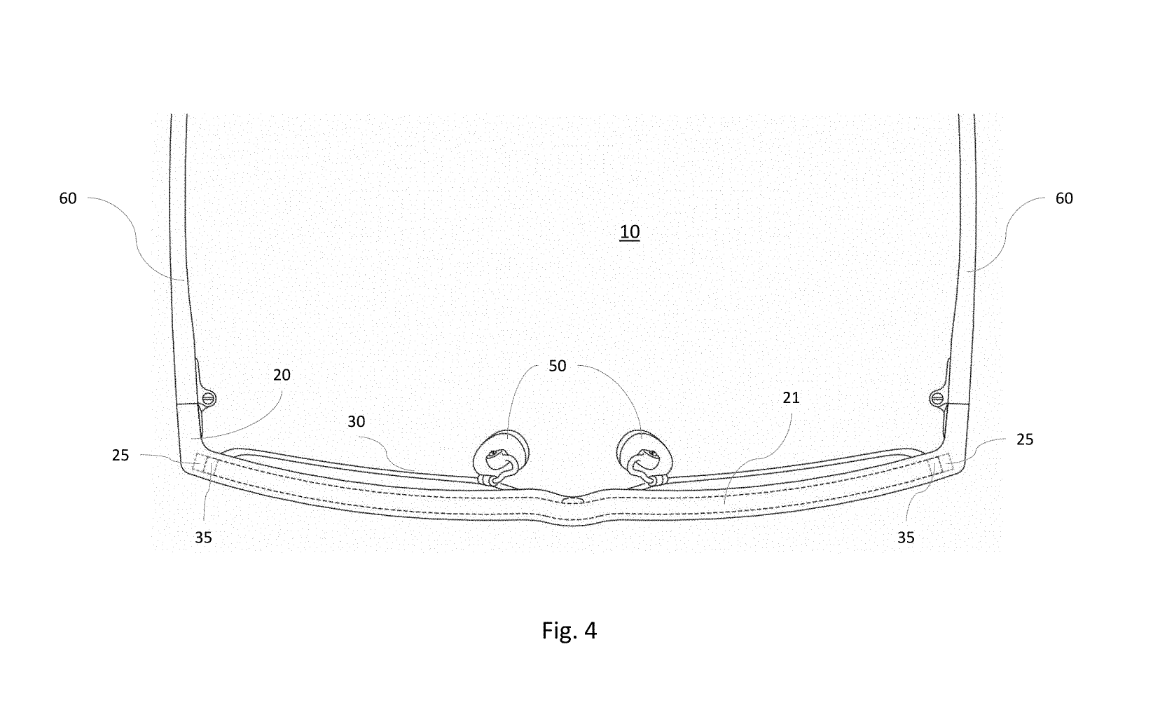

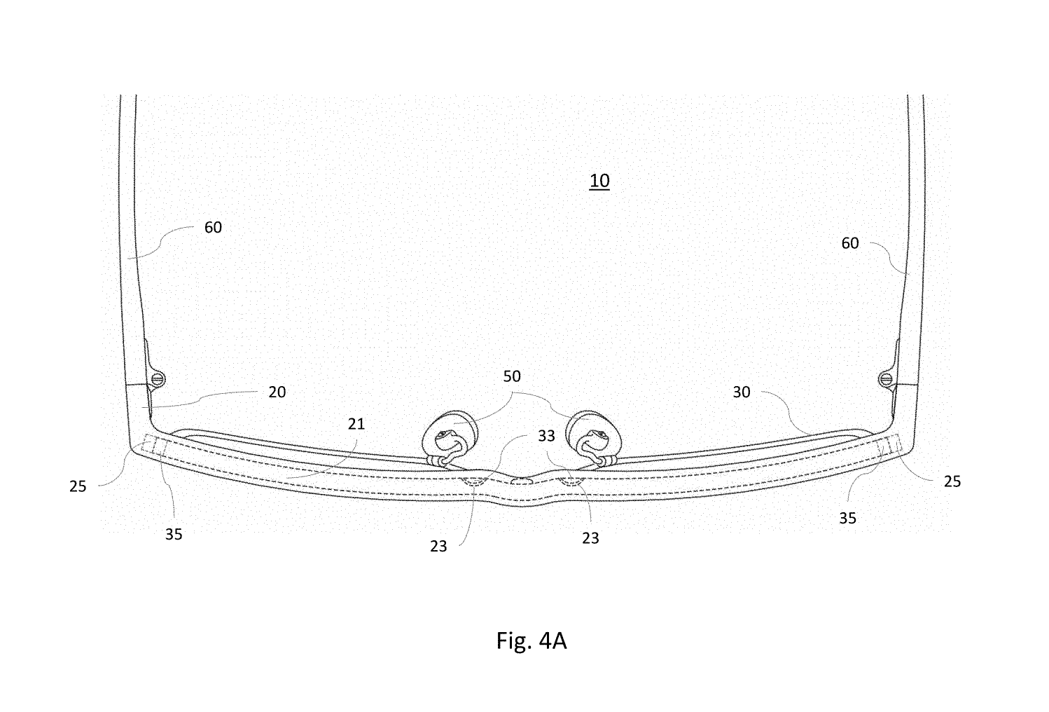

The eyeglass frame section 20 is constructed to accept any one of an interchangeable plurality of lens rim configurations and styles using at least two positioning and retaining elements. First, a channel section or groove 21 is integrally formed in the lower side or edge of the frame section 20, as illustrated in FIGS. 1 and 4. Second, at least one magnetic retaining section 25 is incorporated into the eyeglass frame section 20. In one embodiment, the at least one magnetic retaining section 25 is located such that one side of the at least one magnetic retaining section 25 is located flush with a side of the channel section, as also illustrated in FIG. 1.

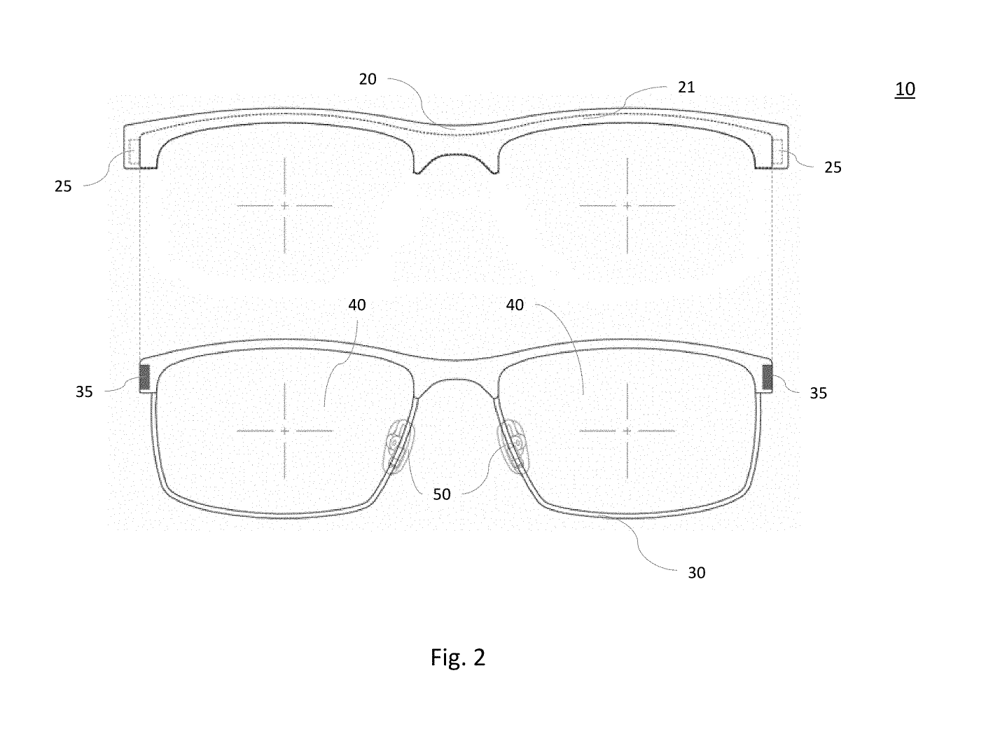

The lens rim 30 is positioned and fits within the eyeglass frame section 20, as shown in FIG. 2 exploded view, with the top side of the lens rim 30 fitting into the channel section 21 of the frame section 20. As shown, the lens rim 30 has an associated at least one magnetic retaining section 35 incorporated into the lens rim 30 such that when the lens rim 30 is properly positioned within the channel section 21 of the frame section 20, the lens rim magnetic retaining section 35 is positioned adjacent to, and providing a magnetic retaining force with the magnetic retaining section 25 incorporated into the frame section 20.

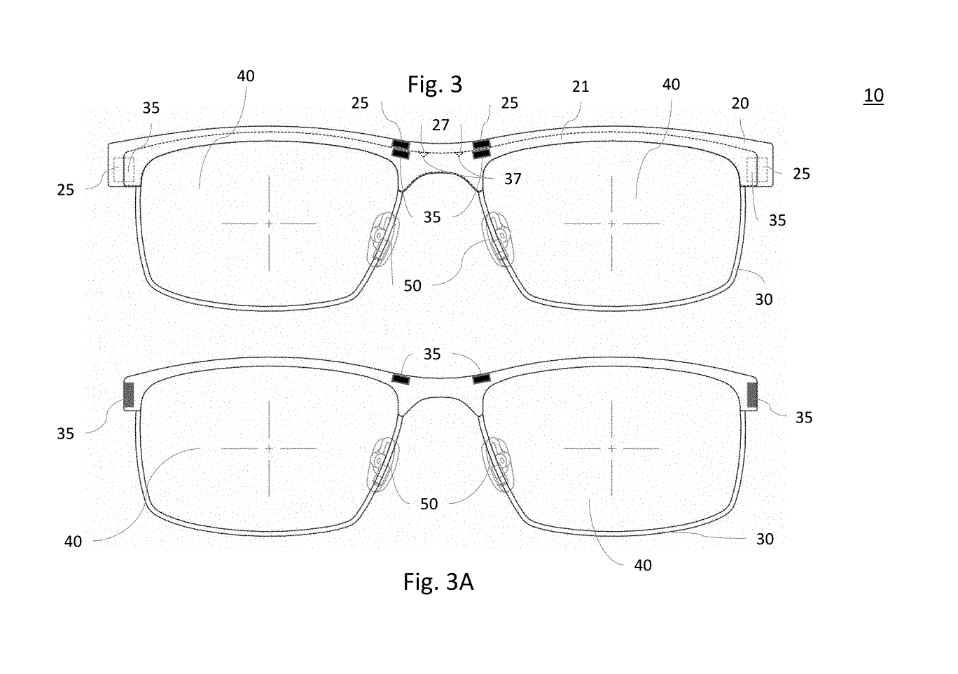

As illustrated in FIGS. 1 and 2, two magnetic retaining sections 25 may be incorporated into the frame section, as shown at the ends of the frame section 20, and an associated two magnetic retaining sections 35 would be incorporated into the lens rim 30 at respective end sections of the lens rim. Additional respective magnetic retaining sections 25 and 35 may be incorporated into the frame section 20 and the lens rim 30, as shown in FIGS. 3 and 3A, for example along the channel or groove 21 of the frame section 20, to provide additional magnetic retaining force to hold the lens rim 30 in position within the frame section 20. In another embodiment, the entire lower side of the frame section 20 along the channel may be magnetic, and similarly the entire upper or top section of the lens rim 30 may similarly be magnetic, thereby providing a single extended section of both the frame section 20 and lens rim 30 creating an extended magnetic attracting force to hold the lens rim 30 in a proper position. Within the frame section 20, either side or edge of the channel 21 may incorporate a magnetic retaining section 25.

Additional stability and positioning alignment between the lens rim 30 and frame section 20 may be provided by including one or more indentations or notches 37 with associated projections or teeth 27 that fit into the indentations 37. FIG. 3 shows an embodiment with two such indentations 37 in the lens rim 30 and projections 27 incorporated into the frame section 20. In other embodiments, additional indentations 37 and projections 27 could be incorporated into the lens rim 30 and frame section 20.

A top view of an embodiment of the interchangeable eyeglass/lens rim set is shown, illustrating the groove 21 formed within the eyeglass frame 20 and the proper positioning of the lens rim 30 within the groove 21. Nose bridge or cushion elements 50 may be integrally formed as part of the lens rim 30, or, as shown in FIGS. 1 through 4, may be separately attached along the inner lens rim section, with the attachment providing some flexibility in the orientation of the nose cushion against the wearer's nose.

In another embodiment, as shown in FIG. 4A, the interchangeable eyeglass/lens rim set may also include one or more ridges 23 formed on the frame 20, and opposing notch sections 33 formed in the lens rim 30. In operation, the eyeglass frame ridge 23 fits or snaps into place into the lens rim notch 33 when the lens rim 30 is pushed into place within the frame channel 21. By providing a notch section 33 and rim ridge 23, the lens rim 30 may be held in place and connected to the eyeglass frame 20 with more stability. In a further modified version of this embodiment, not shown in FIG. 4A, the eyeglass frame could be formed with a ridge while the lens rim could be formed with an opposing notch, thereby reversing where the notch and ridge are located. Either design will work equally well for positioning and holding the lens rim 30 in place within the eyeglass frame 20.

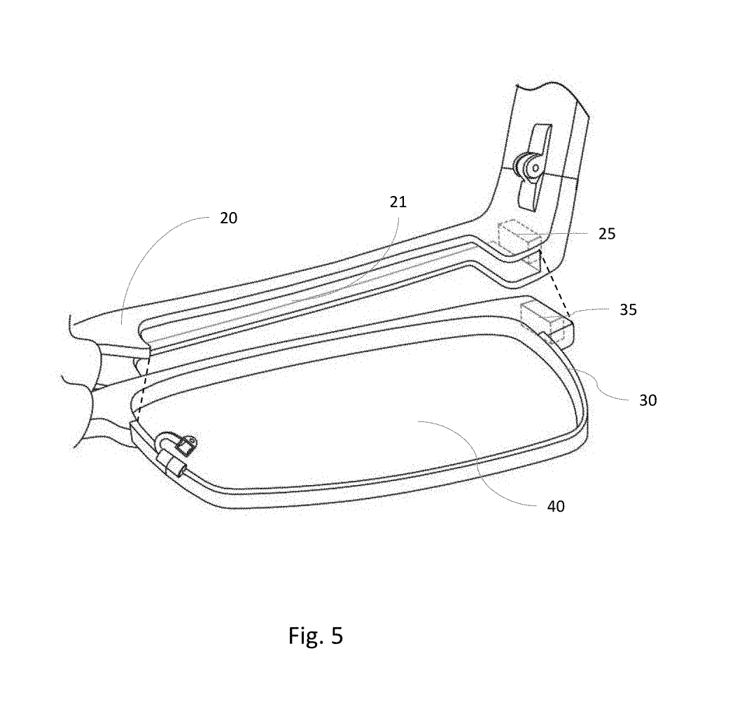

A further detailed view of the eyeglass frame channel 21 is shown in the exploded view of FIG. 5 noting how the lens rim 30 fits into the channel 21 of the eyeglass frame 20 such that the respective magnetic elements 25 and 35 are positioned adjacent to each other to provide the magnetic retaining force to hold the lens rim 30 in proper position within the eyeglass frame 20 and frame channel 21.

While the referenced FIGS. 1 through 5 show the eyeglass frame 20 having a channel section 21, and the lens rim 30 being sized and shaped to fit within the channel section 21, in another embodiment of the invention, the lens rim 30 could be formed having a channel section 31 along the lens rim 30 top edge, and into which an appropriately sized and shaped eyeglass frame 20 could fit. This reversing of the channel section provides an alternative mechanism where the entire lens rim shape and outline is visible, as compared to the alternative design that hides part of the lens rim shape within the eyeglass frame channel 21. This alternative embodiment provides a further means of allowing the wearer to quickly and easily change the appearance of his or her eyeglasses.

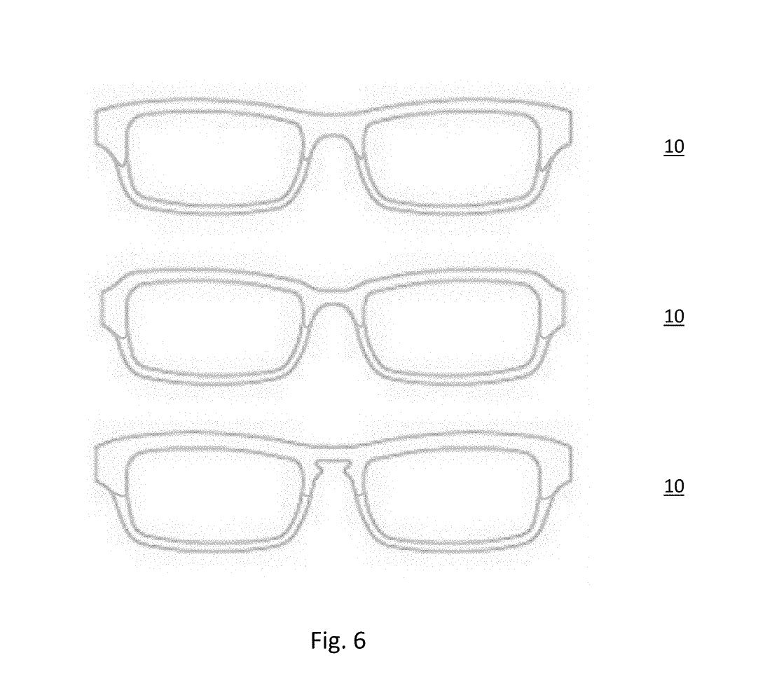

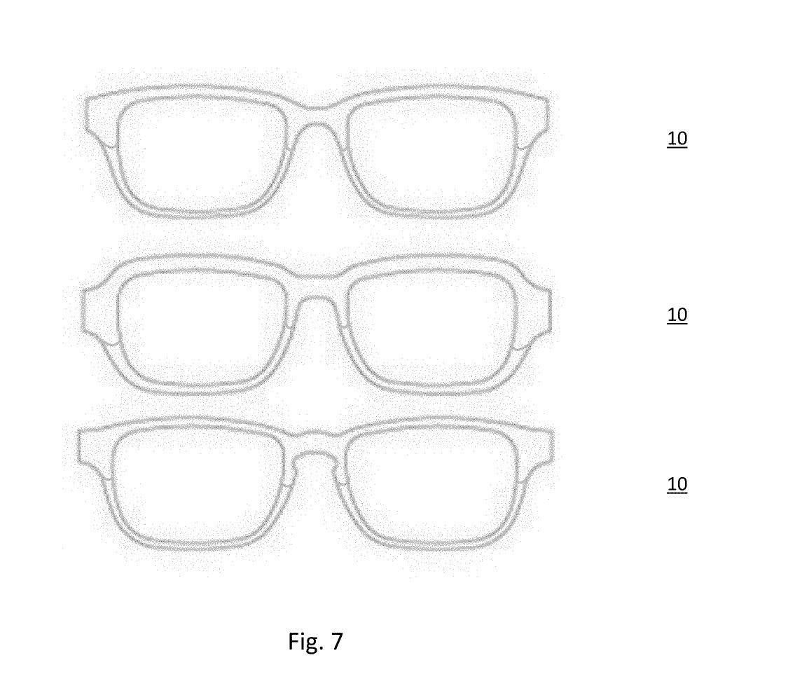

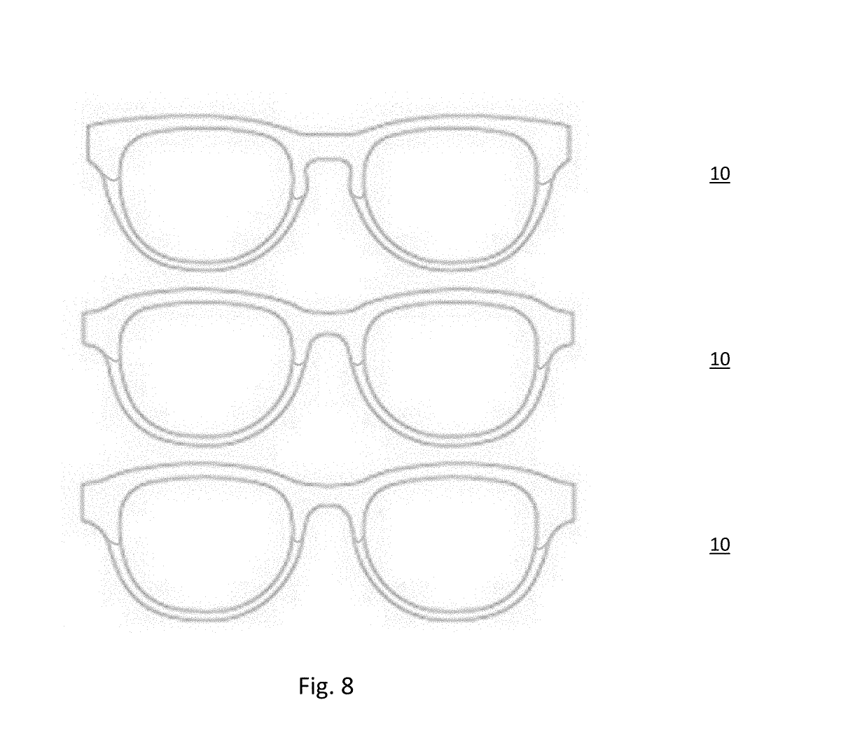

As shown in FIGS. 6, 7, and 8, most any shape or style for the eyeglass lens rim 30 may be substituted and used with the eyeglass frame 20 so long as the lens rim upper side is shaped to fit within the eyeglass frame channel 21. In this manner, a user can readily change his or her style of glasses from Horn-Rim to Wayfarer to Jackie O to Aviators by simply removing the lens rim 30 from the eyeglass frame 20 by a pulling motion against the magnetic forces holding the lens rim in place. FIG. 6 shows three variations of a narrow horn-rim design, while FIG. 7 shows a more traditional and taller lens design for a horn-rim design, including a Jackie O style. Similarly, FIG. 8 shows variations again of still a taller lens set for a horn-rim design. As illustrated, so long as the lens rim 30 fits into the channel 21 of the eyeglass frame 20, the appearance, style and shape of the eyeglasses may be readily altered with the inventive design.

The eyeglass frame 20 and lens rim 30 may be manufactured from a variety of materials. A form of plastic or thermoplastic for the eyeglass frame provides a resilient, yet pliable material that allows the lens rim 30 to easily be positioned within the channel 21 that is formed in the frame 20. To provide a wide range of appearances and looks for the assembled eyeglasses, the lens rim 30 may be manufactured from a plastic material, a composite material, wood, or a metal, such as aluminum or titanium. Other metals or different types of thermoplastics may also be used with equal utility and provide a wide variety of styles, appearances and looks. Of course, the eyeglass frame 20 and lens rim 30, as well as the lenses 40 themselves, may be manufactured in most any color or combination of colors that suit users and customers.

For further ability to alter the style and look of the eyeglasses, the earpieces or temples 60 may be connected to the eyeglass frames 20 using a releasable hinge mechanism as disclosed in U.S. Pat. No. 8,317,318 (the '318 patent“) assigned to Silver Global Manufacturing, LLC. According to the '318 patent, the eyeglass frame 30 has male bayonet clips incorporated at each end of the eyeglass frame 30, and each of the temple members 60 has an integrally formed female bayonet clip at the end of the temple member so that there is complemental engagement of the temple member bayonet clip with an opposing eyeglass frame bayonet clip. Using such a design for interchanging the temple pieces, the wearer of the inventive eyeglasses has complete and readily changeable control over the entire style, design, color, look and feel of his or her eyeglasses.

Certain preferred embodiments and examples of the inventive interchangeable eyeglass frame and lens rim design using a structural channel and magnetic retaining forces have been described herein. It is to be understood that various modifications may be made to these described embodiments without departing from the spirit and scope of the invention. More particularly while certain exemplary embodiments of the inventive interchangeable eyeglass frame and lens rim design have been disclosed and described, there may be other design modifications, including as proposed herein, placement of the magnetic sections, particular shape of the channel section, and materials used to manufacture the eyeglass frame and lens rim that operate in the same manner as the claimed inventive design.

All such alternative magnet placements, channel shapes, or alternative manufacturing materials and designs are considered equivalent configurations, modifications and other embodiments of the claimed invention, and are therefore intended to be, and are deemed within the scope of the above description and within the following claims. Those having ordinary skill in the art and access to the teachings provided herein will recognize additional modifications, applications, embodiments and substitution of equivalents all of which are within the scope of the invention. Accordingly, the invention is not to be considered as limited by the specific foregoing description or particular illustrated embodiments.

What I claim as my invention is:

1. An interchangeable eyeglass lens rim and eyeglass frame system, said system comprising:
(a) a lens rim encircling and holding a pair of eyeglass lenses;
(b) at least one magnetic element incorporated within said lens rim;
(c) an eyeglass frame component having a channel section along one edge into which said lens rim fits; and
(d) at least one magnetic element located within said eyeglass frame component such that said at least one magnetic element attracts said at least one magnetic element within said lens rim, and thereby firmly maintains said lens rim in position within said channel section and against said eyeglass frame component.

2. An eyeglass frame and lens rim set wherein said lens rim is interchangeable, said frame and lens rim set comprising:
(a) an eyeglass frame component having a cut out section along a lower edge;
(b) at least a first magnetic section incorporated within said cut out section of said eyeglass frame;
(c) a lens rim component fully encircling a pair of eyeglass lenses;
(d) at least a second magnetic section incorporated along a top edge of said lens rim component;
whereby with said lens rim component properly positioned within said eyeglass frame component and within said cut out section, said second magnetic section attracts said first magnetic section and holds said lens rim component firmly in place with said eyeglass frame component in said cut out section.

3. The interchangeable eyeglass lens rim and eyeglass frame system, as recited in claim 1, further comprising at least one notch section within said lens rim, and an associated at least one ridge section within said eyeglass frame component such that when said lens rim is in proper position within said eyeglass frame channel section, said associated at least one ridge section fully engages within said at least one notch section within said lens rim.

4. The interchangeable eyeglass lens rim and eyeglass frame system, as recited in claim 2, further comprising at least one notch section within said lens rim, and an associated at least one ridge section within said eyeglass frame component such that when said lens rim is in proper position within said eyeglass frame channel section, said associated at least one ridge section fully engages within said at least one notch section within said lens rim.

5. The interchangeable eyeglass lens rim and eyeglass frame system, as recited in claim 1, wherein said lens rim is manufactured from a thermoplastic material.

6. The interchangeable eyeglass lens rim and eyeglass frame system, as recited in claim 1, wherein said lens rim is manufactured from a metallic material.

7. The interchangeable eyeglass lens rim and eyeglass frame system, as recited in claim 6, wherein said lens rim is manufactured from a material consisting of at least one of aluminum, titanium, or steel.

8. The interchangeable eyeglass lens rim and eyeglass frame system, as recited in claim 1, wherein said at least one magnetic element incorporated within said lens rim is a top section of said lens rim above each lens.

9. The interchangeable eyeglass lens rim and eyeglass frame system, as recited in claim 1, further comprising at least one indentation within said lens rim, and an associated at least one projection within said eyeglass frame component such that when said lens rim is in proper position within said eyeglass frame channel section, said at least one indentation and associated at least one projection properly positions said lens rim within said eyeglass frame component.

10. A set of eyeglasses having an interchangeable lens rim and lenses, said set of eyeglasses comprising:
(a) an eyeglass frame having a channel section along a lower edge;
(b) a first magnetic section incorporated into said eyeglass frame channel section;
(c) a lens rim fully encircling a set of eyeglass lenses;
(d) a second magnetic section incorporated into said lens rim;
whereby when said lens rim is in a proper position within said eyeglass frame channel section, said second magnetic section attracts said first magnetic section, and maintains said lens rim in position against said eyeglass frame.

11. The set of eyeglasses having interchangeable lens rim and lenses, as recited in claim 10, further comprising a plurality of notches integrally formed within said lens rim, and an associated plurality of ridges integrally formed within said eyeglass frame component such that when said lens rim is in proper position within said eyeglass frame channel section, said associated plurality of ridges fully engage within said plurality of notches within said lens rim.

12. The interchangeable eyeglass lens rim and eyeglass frame system, as recited in claim 10, wherein said lens rim is manufactured from a thermoplastic material.

13. The interchangeable eyeglass lens rim and eyeglass frame system, as recited in claim 10, wherein said lens rim is manufactured from a metallic material.

14. The interchangeable eyeglass lens rim and eyeglass frame system, as recited in claim 13, wherein said lens rim is manufactured from a material consisting of at least one of aluminum, titanium, or steel.

15. An interchangeable eyeglass lens rim and eyeglass frame system, said system comprising:
a. a lens rim encircling and holding a pair of eyeglass lenses, said lens rim having a channel section along said lens rim upper side;
b. at least one magnetic element incorporated within said lens rim;
c. an eyeglass frame component having a lower side that securely fits into said channel section of said lens rim; and
d. at least one magnetic element located within said eyeglass frame component such that said at least one magnetic element attracts said at least one magnetic element within said lens rim, and thereby firmly maintains said eyeglass frame component in position within said channel section and against said lens rim.

16. The interchangeable eyeglass lens rim and eyeglass frame system, as recited in claim 15, wherein said lens rim is manufactured from a thermoplastic material.

17. The interchangeable eyeglass lens rim and eyeglass frame system, as recited in claim 15, wherein said lens rim is manufactured from a metallic material.

18. The interchangeable eyeglass lens rim and eyeglass frame system, as recited in claim 15, further comprising a plurality of indentations integrally formed within said lens rim, and an associated plurality of projections integrally formed within said eyeglass frame component such that when said lens rim is in proper position within said eyeglass frame channel section, said associated plurality of projections fully engage within said plurality of indentations within said lens rim.
